(19) United States
(12) Patent Application Publication
(10) Pub. No.: US 2025/0017997 A1
(43) Pub. Date: Jan. 16, 2025

(54) USE OF EXTRACT IN MANUFACTURING A PHARMACEUTICAL OR NON-PHARMACEUTICAL COMPOSITION FOR ENHANCING THE ACTIVITY OF MITOCHONDRIA

(71) Applicant: TAIWAN MITOCHONDRION APPLIED TECHNOLOGY CO., LTD., Zhubei City (TW)

(72) Inventors: Shun-Chieh YANG, Zhubei City (TW); Li-Hsin YAO, Zhubei City (TW)

(21) Appl. No.: 18/220,043

(22) Filed: Jul. 10, 2023

(51) Int. Cl. A61K 36/355; A61K 31/216; A61P 3/00

(52) U.S. Cl. CPC A61K 36/355; A61K 31/216; A61P 3/00; A61K 2236/31; A61K 2236/331; A61K 2236/51

(57) ABSTRACT

The present disclosure provides a use of an extract in manufacturing a pharmaceutical or non-pharmaceutical composition for enhancing the activity of mitochondria. In the present disclosure, the activity-enhanced mitochondria can maintain their function and activity when being stressed to ensure normal work of cells without affecting by external or internal stress.

### BACKGROUND

### 1. Technical Field

This disclosure relates to a use of extract in manufacturing a pharmaceutical or non-pharmaceutical composition for enhancing the activity of mitochondria, and more particularly, to a use of Lonicera Japonica extract in manufacturing a pharmaceutical or non-pharmaceutical composition for enhancing the activity of mitochondria.

### 2. Related Art

Mitochondria (called “mitochondrion” in singular form) are places where oxidative phosphorylation (OXPHOS) and adenosine triphosphate (ATP) synthesis occur. Since ATP is used as a source of energy in a cell, mitochondria are described as the powerhouse of the cell. In addition to generate energy required by the cell, mitochondria also participate in cell division, cell signaling and apoptosis, and have the ability to control the cell-division cycle.

However, some of side products generated in the oxidative phosphorylation are harmful to mitochondria, such as reactive oxygen species (ROS), including superoxide anion (O₂·⁻), perhydroxyl radical (HO₂·), hydrogen peroxide (H₂O₂) and the like. ROS have strong biochemical reactivity and are easy to cause oxidative damage to cells or mitochondria. The damaged mitochondria have adverse effects on cell energy supply, cell growth and the like. After accumulating for a long time, the severely damaged mitochondria would release cytochrome c (Cyt c), caspase, procaspase-2, procaspase-3, procaspase-8, procaspase-9 and the like, and these may trigger the collapse of the mitochondria. The severely damaged mitochondria would also release apoptosis-related signaling factors, including proteases of B cell lymphoma/leukemia-2 (Bcl-2) family, apoptotic protease activating factor-1 (Apaf-1), p53, serine protease Omi/HtrA2 and the like, thereby triggering the collapse of the mitochondria. Therefore, how to enhance the ability of mitochondria to cope with stress, protect and repair mitochondria to maintain their functions and reduce the collapse of mitochondria have become important issues.

### SUMMARY

According to one embodiment of the present disclosure, a use of an extract in manufacturing a pharmaceutical or non-pharmaceutical composition for enhancing the activity of mitochondria is provided, wherein the extract is Lonicera Japonica extract.

### BRIEF DESCRIPTION OF THE DRAWINGS

The present disclosure will become more fully understood from the detailed description given hereinbelow and the accompanying drawings which are given by way of illustration only and thus are not limitative of the present disclosure and wherein:

FIG. 1 shows the HPLC chromatogram of the Lonicera Japonica extract;

FIG. 2 shows the NIR spectrum of the Lonicera Japonica extract processed using Savitsky-Golay smoothing;

FIG. 3 shows the result of the cytotoxicity test for the Lonicera Japonica extract

with different concentrations;

FIG. 4 shows the oxygen consumption of the mitochondria for overcoming proton leakage;

FIG. 5 shows the oxygen consumption of the mitochondria for ATP production;

FIG. 6 shows the oxygen consumption of the mitochondria for the spare respiration;

FIG. 7 shows the oxygen consumption of the mitochondria for the maximal respiration; and

FIG. 8 shows the ATP coupling efficiency of the mitochondria.

### DETAILED DESCRIPTION

In the following detailed description, for purposes of explanation, numerous specific details are set forth in order to provide a thorough understanding of the disclosed embodiments. According to the description, claims and the drawings disclosed in the specification, one skilled in the art may easily understand the concepts and features of the present disclosure. The following embodiments further illustrate various aspects of the present disclosure, but are not meant to limit the scope of the present disclosure.

Lonicera Japonica, also known as honeysuckle, is a perennial and semi-evergreen twining vine in the genus Lonicera of the family Caprifoliaceae, and occurs in the subtropics and temperate zone of North America, Europe, Asia and North Africa. The buds and petals of Lonicera Japonica comprise ingredient such as chlorogenic acid, isochlorogenic acid, ginnol, β-sitosterol, stigmasterol, β-sitosterol-β-D-glucoside, stigmasterol-D-glucoside and the like, and volatile oil such as linalool, cis-2,6,6-trimethyl-2-vinyl-5-hydroxytetrahydrofuran, ethylpalmitate, 1,1-bicyclohexyl, 3-methyl-2-(2-pentenyl)-2-cyclopenten-1-one, trans-trans-farnesol, ethyllinolemate, ß-cubebene, cis-3-hexen-1-ol, α-erpineol, benzylbenzoate, 2-methyl-1-butanol, benzyl alcohol, phenethyl alcohol, cis-linalool oxide, eugenol and carvacrol.

The main active ingredient of Lonicera Japonica is chlorogenic acid, and its bioactivity comprises inhibiting hyaluronidase and glucose-6-phosphate, scavenging free radicals, anti-lipid peroxidation, antimutagenicity, protecting liver and gallbladder, antibacterial activity, antiviral activity and antispasmodic activity. In the traditional Chinese medicine, Lonicera Japonica has functions of dispelling heat, detoxifying and treating wind-heat and pharmacological effects such as antibacterial activity, anti-inflammatory, antiviral activity, and blood lipid lowering, and is used to treat symptoms such as carbuncle and furuncle, throat obstruction, toxic dysentery, wind heat and fever.

The extract of one embodiment of the present disclosure is Lonicera Japonica extract, which may be obtained by extracting the petals of Lonicera Japonica with water and then drying. Specifically, first, the petals of Lonicera Japonica are washed with water three times. Then, the petals of the Lonicera Japonica are extracted with water at 60° C. to 80° C. three times, the extraction volume ratio of the petals to water is about 1:3, and the extraction time of each is three hours. The extracted solution is cooled to room temperature. Then, the cooled extracted solution is sterilized at 80° C. for 20 hours and then spray-dried to obtain the powdered extract. Finally, the powdered extract is filtered with an 80-mesh sieve to obtain yellowish brown powdered Lonicera Japonica extract. In other embodiments, the Lonicera Japonica extract may also be obtained by extracting with other polar solvents, and the petals may also be chopped or ground before extracting.

The Lonicera Japonica extract, which is obtained by extracting with water as described above, comprises chlorogenic acid. The concentration of chlorogenic acid in the Lonicera Japonica extract may be 20 wt % to 30 wt %. In some embodiments, the concentration of chlorogenic acid in the Lonicera Japonica extract may be 25 wt %. In other embodiments, the Lonicera Japonica extract obtained by extracting with water may comprise about 25 wt % of chlorogenic acid, 17.7 wt % of secologanic acid and 0.5 wt % of galuteolin.

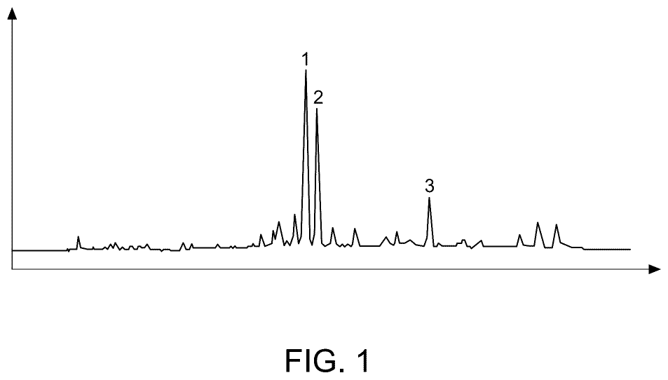

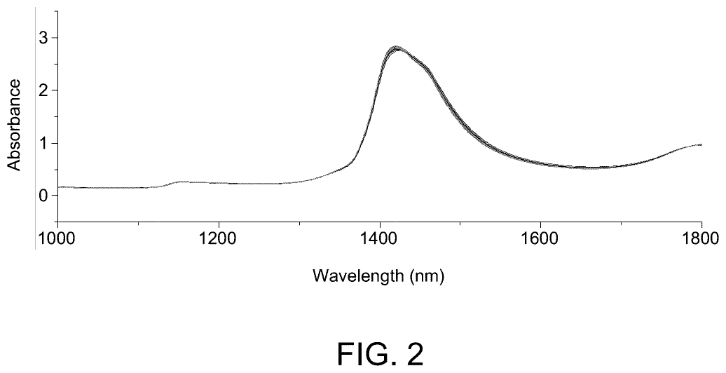

The Lonicera Japonica extract is analyzed by HPLC, and the results are shown in Table 1 and FIG. 1. FIG. 1 shows the HPLC chromatogram of the Lonicera Japonica extract, and the numbers 1 to 3 in Table 1 correspond to the peaks 1 to 3 in FIG. 1. The Lonicera Japonica extract is analyzed by infrared spectrophotometer and processed using Savitsky-Golay smoothing, and the results are shown in FIG. 2. FIG. 2 shows the NIR spectrum of the Lonicera Japonica extract processed using Savitsky-Golay smoothing.

TABLE 1

| Number | Ingredient | Average concentration | | weight percentage |
|---|---|---|---|---|
| 1 | Secologanic acid | 0.3795 | mg/mL | 17.7% |
| 2 | Chlorogenic acid | 0.5340 | mg/mL | 25% |
| 3 | Galuteolin | 10.69 | μg/mL | 0.5% |

In some embodiments of the present disclosure, the activity of the mitochondria may be enhanced by providing 200 μg/mL to 500 μg/mL of the Lonicera Japonica extract to cells. More specifically, the spare respiratory capacity, the maximal respiratory capacity, and the ATP production of the mitochondria may be enhanced, the proton leakage of the mitochondria may be reduced, the ATP coupling efficiency of the mitochondria may be enhanced, and the BHI of the mitochondria may be enhanced. In another embodiment, the concentration of the Lonicera Japonica extract may be 200 μg/mL to 250 μg/mL. In other embodiments, the concentration of the Lonicera Japonica extract may be 250 μg/mL to 500 μg/mL.

As a manner for providing the Lonicera Japonica extract to cells, for example, the Lonicera Japonica extract may be taken by oral administration. In the case of providing the Lonicera Japonica extract to cells by oral administration, the effective dose of the Lonicera Japonica extract may be 2.162 g to 5.406 g. The effective dose is obtained according to the following conversion equation: (effective dose in human)=(effective dose in cell experiment)×(body weight of mice)×(conversion coefficient)×(body weight of human). The conversion coefficient is obtained from the conversion coefficient table. For example, when the body weight of mice is 20 g and the body weight of human is 60 kg, the conversion coefficient is 9.01. In another embodiment, the effective dose of the Lonicera Japonica extract may be 2.162 g to 2.703 g. In other embodiments, the effective dose of the Lonicera Japonica extract may be 2.703 g to 5.406 g.

To make the oral administration more convenient, the Lonicera Japonica extract may be made into processed foods provided in, for example, liquid form, solid form, powder form, granular form, paste form, or gel form. In some embodiments of the present disclosure, without affecting the effect and the purpose of the present disclosure, the processed food of the Lonicera Japonica extract may also comprise other ingredients or additives, such as a carrier, a diluent, an adjuvant, an excipient, or a flavor enhancer. The excipient may make the formulation convenient and practical, and the flavor enhancer may improve the flavor of the formulation.

For example, the excipient may be starch, such as wheat starch, rice starch, corn starch, potato starch, dextrin, cyclodextrin, and the like; crystalline cellulose; saccharide, such as lactose, glucose, sugar, reduced maltose, cerealose, oligofructose, galactooligosaccharide, and the like; or glycitol, such as sorbitol, erythritol, xylitol, lactitol, mannitol, and the like.

For example, the flavor enhancer may be fruit extract, such as longan extract, lychee extract, grapefruit extract, and the like; fruit juice, such as apple juice, orange juice, lemon juice, and the like; essence, such as peach essence, plum essence, yogurt essence, and the like; sweetener, such as acesulfame potassium, sucralose, erythritol, oligosaccharide, mannose, xylitol, isomerized sugar, and the like; acid flavoring, such as citric acid, malic acid, tartaric acid, gluconate, and the like; or tea ingredient, such as green tea, oolong tea, banaba tea, eucommia tea, tieguanyin tea, coix tea, jiaogulan tea, zizania latifolia tea, kelp tea, and the like.

Moreover, the composition of the Lonicera Japonica extract according to the present disclosure may be a pharmaceutical composition or a non-pharmaceutical composition and may also be a health supplement. The Lonicera Japonica extract or the composition comprising the Lonicera Japonica extract may also be encapsulated in a capsule for convenient oral administration. The Lonicera Japonica extract or the composition comprising the Lonicera Japonica extract may be encapsulated in a hard capsule in a dried powder form and may also be encapsulated in a soft capsule in a liquid form, suspension form, paste form, powder form, or granular form.

The oil in the soft capsule for dissolving the Lonicera Japonica extract may be, for example, avocado oil, almond oil, flaxseed oil, fennel oil, perilla frutescens oil, olive oil, olive squalene, sweet orange oil, orange roughy oil, sesame oil, garlic oil, cocoa butter, pumpkin seed oil, chamomile oil, carrot oil, cucumber oil, tallow fatty acid, kukui nut oil, lingonberry seed oil, brown rice germ oil, rice bran oil, wheat germ oil, safflower oil, shea butter, liquid shea butter, perilla oil, soybean oil, evening primrose oil, camellia oil, corn oil, rapeseed oil, saw palmetto extract oil, coix oil, peach kernel oil, celery seed oil, castor oil, sunflower oil, grapeseed oil, borage oil, macadamia nut oil, meadowfoam oil, cottonseed oil, peanut oil, turtle oil, mink oil, egg yolk oil, fish oil, palm oil, palm-kernel oil, wood wax oil, coconut oil, long-chain/medium-chain/short-chain triglyceride, diglyceride, butter, lard, squalene, squalane and pristane and hydrides thereof.

In addition, several food additives approved for use, such as colorant, preservative, tackifier, binder, disintegrant, dispersant, stabilizer, gelatinizer, antioxidant, surfactant, preservative, and pH control agent, may be added to the processed food of the Lonicera Japonica extract.

The following demonstrates the effect of enhancing the activity of the mitochondria using the Lonicera Japonica extract of the present disclosure. The Lonicera Japonica extract used in the following experiment is obtained by extracting the petals of Lonicera Japonica with water and then drying as described above, and the Lonicera Japonica extract comprises at least chlorogenic acid. The cells used in the following experiment are skeletal muscle cells (C2C12). The cell culture is performed in DMEM with 10% fetal bovine serum (FBS). The cell subculture is described as follows. First, the cells are cultured to a certain amount, and then the culture medium is removed. The cells are rinsed with phosphate buffered saline (PBS) twice. Then, trypsin is added to react with the cells at 37° C. for 5 minutes, and then the culture medium is added to stop the reaction of trypsin. Then, the mixture is centrifuged at 300 g for 5 minutes to remove the supernatant and resuspended with the culture medium. Finally, the cells are transferred to a 175 T flask for subsequent experiments, and the cell count in the 175 T flask is 1×10⁶ cells.

Experiment 1: The cytotoxicity test for the Lonicera Japonica extract

First, the cytotoxicity test for the Lonicera Japonica extract is conducted. Alamar blue is a cell viability assay reagent. In the Alamar blue cell viability assay kit, resazurin is a redox indicator, which is a nontoxic, cell-permeable, weakly fluorescent, and deep blue dye. Upon entering living cells, resazurin is reduced to resorufin, a compound that is pink and highly fluorescent, due to the reducing environment in the living cells. The cell viability may be evaluated by detecting the absorbance or fluorescence of resorufin in the cells. The higher absorbance or fluorescence of resorufin indicates the higher cell viability. High viability means healthy cells and a high proliferation ability. When the cells have a high proliferation ability, the amount of cells increases. Therefore, Alamar blue may be used as an indicator of cytotoxicity to reflect cell viability and cell proliferation.

The procedure for the cytotoxicity test of the Lonicera Japonica extract is described as follows. On the first day, the cells are cultured in a 96-well plate with a total volume of 200 μL and 10000 cells per well for one day. On the second day, the Lonicera Japonica extract is added, and the concentrations of the Lonicera Japonica extract in each well are 50, 100, 200, 250, 500, and 1000 μg/mL. The cells are incubated at 37° C. for one day. On the third day, the cytotoxicity test is conducted with Alamar blue. In detail, Alamar blue is prepared to a solution of 10 wt % in a dark environment, added to the 96-well plate with 100 μL per well, and incubated with the cells at 37° C. for 3 to 4 hours. Then, the absorbance and fluorescence are measured by ELISA reader (OD530/590), and the cell viability after being treated with the Lonicera Japonica extract is obtained to represent the cytotoxicity of the Lonicera Japonica extract.

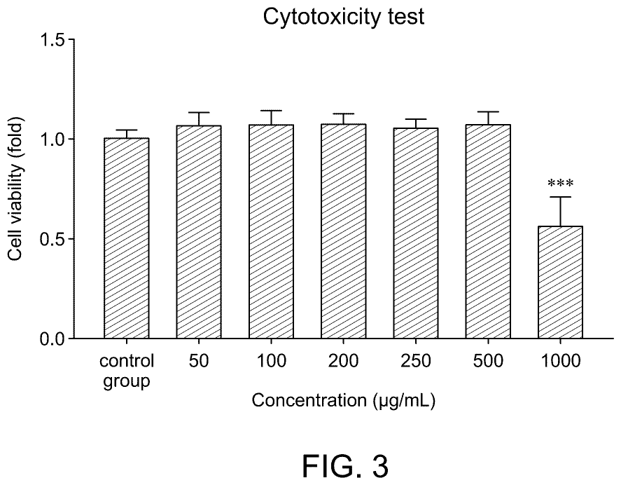

FIG. 3 shows the result of the cytotoxicity test for the Lonicera Japonica extract with different concentrations. The control group is the cells not treated with the Lonicera Japonica extract, the vertical axis is the cell viability relative to the control group, and the symbol “***” (P<0.01) means significantly different relative to the control group.

As shown in FIG. 3, the Lonicera Japonica extract less than 500 μg/mL have no effect on cell viability. This indicates that the Lonicera Japonica extract less than 500 μg/mL have no cytotoxicity. Accordingly, 200, 250, and 500 μg/mL of the Lonicera Japonica extract are selected as Examples 1 to 3 of the present disclosure for the subsequent experiments.

Experiment 2: enhancing the activity of the mitochondria by the Lonicera Japonica extract

Next, the experiment of enhancing the activity of the mitochondria by the Lonicera Japonica extract is conducted. In the experiment, tert-butyl hydroperoxide (t-BHP) is used as a substance that induces cellular oxidative stress damage and aging and inhibits the activity of the mitochondria.

The experimental procedure for enhancing the activity of the mitochondria by the Lonicera Japonica extract is described in detail as follows. On the first day, the cells are cultured with culture medium in a 24-well plate for Seahorse XF analysis with a total volume of 100 μL and 25000 cells per well for 4 hours, and then 150 μL of the culture medium is added and incubated for one day. On the second day, the Lonicera Japonica extract is added, and the concentrations of the Lonicera Japonica extract in each well are 200, 250, and 500 μg/mL with a total volume of 250 μL in each well. The cells are incubated with the Lonicera Japonica extract for one day. On the third day, the medium is replaced with fresh culture medium, 100 μM of t-BHP is added to each well and reacted with the cells for 1 hour, and then the culture medium in the well is replaced with 675 μL of the medium for measuring, a DMEM medium without FBS, and incubated in an incubator without CO₂ for 1 hour. Then, the oxygen consumption of the cells in the well is measured by a Seahorse XF analyzer.

The principle and procedure of Seahorse XF analyzer are described as follows. First, the basal respiration of cells is measured. Then, a ATP synthesis inhibitor is added to inhibit the mitochondria to synthesize ATP, and the reduction of the oxygen consumption is equal to the oxygen consumption for ATP production. Then, an anti-coupler in a proper concentration, which causes no damage to the electron transport chain in the inner mitochondrial membrane, is added to evaluate the maximal respiration of the mitochondria. Finally, an electron transport chain inhibitor is added to totally stop the respiration in the mitochondria, and the background is measured, which is equal to the non-mitochondrial respiration. The oxygen consumption of the basal respiration of the mitochondria is equal to the oxygen consumption of the basal respiration of cells minus the oxygen consumption of the non-mitochondrial respiration. The oxygen consumption for overcoming proton leakage is equal to the oxygen consumption of the basal respiration of the mitochondria minus the oxygen consumption of mitochondria for ATP production. The oxygen consumption of the spare respiration is equal to the oxygen consumption of the maximal respiration minus the oxygen consumption of the basal respiration of the mitochondria. The ATP coupling efficiency is equal to the oxygen consumption of the mitochondria for ATP production divided by the oxygen consumption of the basal respiration of the mitochondria.

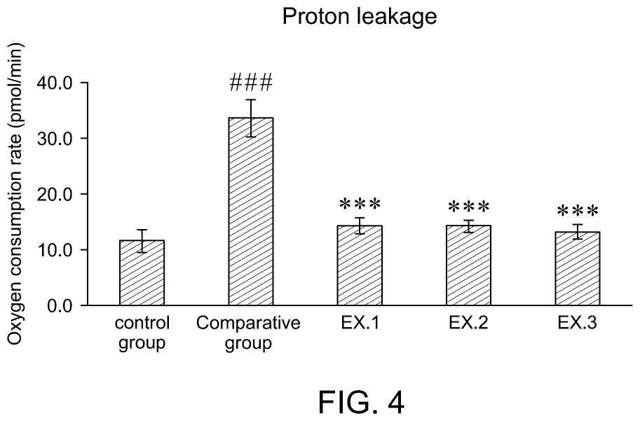

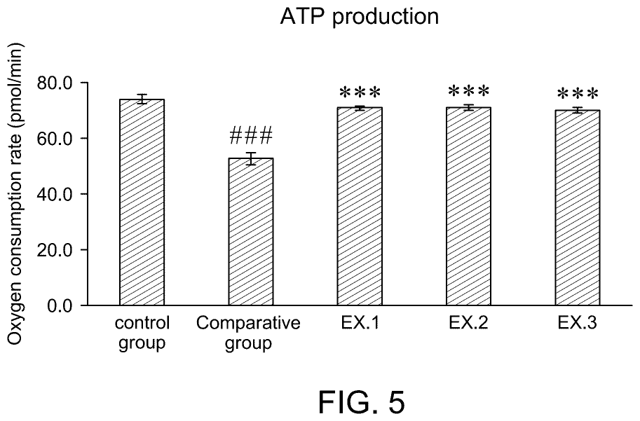

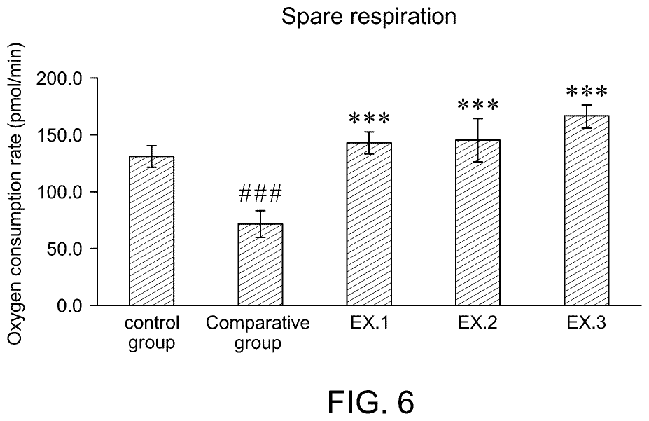

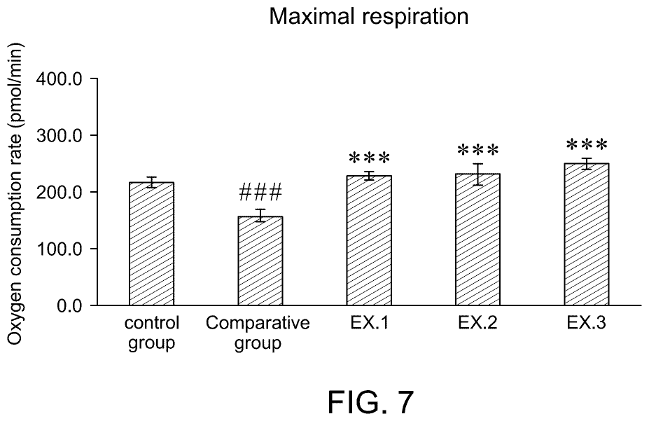

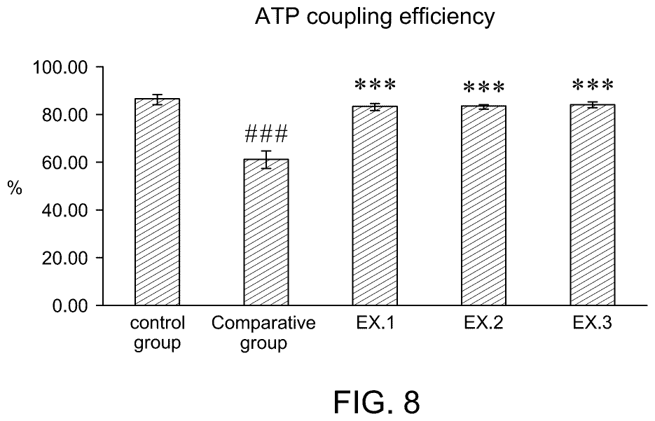

The results are shown in Table 2 and FIGS. 4 to 8, FIG. 4 shows the oxygen consumption of the mitochondria for overcoming proton leakage; FIG. 5 shows the oxygen consumption of the mitochondria for ATP production; FIG. 6 shows the oxygen consumption of the mitochondria for the spare respiration; FIG. 7 shows the oxygen consumption of the mitochondria for the maximal respiration; and FIG. 8 shows the ATP coupling efficiency of the mitochondria. The control group (Con.) is the cells not treated with t-BHP and the Lonicera Japonica extract, the comparative group (Comp.) is the damaged cells treated with 100 μM of t-BHP but no the Lonicera Japonica extract, and the experimental groups (Ex.) are the cells treated with the Lonicera Japonica extract of Examples 1 to 3, respectively, and then treated with 100 μM of t-BHP. In FIGS. 4 to 7, the vertical axis is the oxygen consumption in pmol per minute. In FIG. 8, the vertical axis is the ATP coupling efficiency in percentage (%). Symbol “***” (P<0.001) means significantly different relative to the comparative group, and symbol “###” (P<0.001) means significantly different relative to the control group.

TABLE 2

| | Lonicera | | | | | | Non- | ATP |
|---|---|---|---|---|---|---|---|---|
| | Japonica | Basal | Proton | ATP | Maximal | Spare | mitochondrial | coupling |
| | extract | respiration | leakage | production | respiration | respiration | respiration | efficiency |
| Unit | μg/mL | pmol/min | | | | | | % |
| Con. | — | 85.5 ± 3.04 | 11.6 ± 2.04 | 73.9 ± 1.55 | 216.6 ± 9.42 | 131.1 ± 9.26 | 14.5 ± 3.04 | 86.5 ± 1.98 |
| Comp. | — | 86.1 ± 1.13 | 33.5 ± 3.35 | 52.7 ± 2.23 | 157.6 ± 10.83 | 71.5 ± 11.60 | 13.9 ± 1.13 | 61.2 ± 3.39 |
| Ex. 1 | 200 | 85.0 ± 2.08 | 14.2 ± 1.45 | 70.8 ± 0.69 | 228.0 ± 7.44 | 143.0 ± 9.17 | 15.0 ± 2.08 | 83.4 ± 1.32 |
| Ex. 2 | 250 | 85.1 ± 1.57 | 14.2 ± 1.12 | 70.9 ± 1.07 | 230.6 ± 18.92 | 145.5 ± 19.27 | 14.9 ± 1.57 | 83.4 ± 1.09 |
| Ex. 3 | 500 | 83.0 ± 1.76 | 13.1 ± 1.27 | 70.0 ± 0.82 | 249.8 ± 9.63 | 166.7 ± 10.20 | 17.0 ± 1.76 | 84.3 ± 1.24 |

As shown in FIG. 4, in terms of the oxygen consumption of the mitochondria for overcoming proton leakage, the comparative group is higher than the control group. This means that in the comparative group, the inner membrane of the mitochondria is damaged so that more oxygen is needed to overcome the proton leakage. In contrast, the experimental groups treated with the Lonicera Japonica extract of Examples 1 to 3 are less than the comparative group and close to the control group. This means that the activity of the mitochondria in the experimental groups is enhanced by the Lonicera Japonica extract, and also means that the Lonicera Japonica extract is able to protect and repair the mitochondria under oxidative stress so that the inner membrane of the mitochondria is less damaged.

As shown in FIG. 5, in terms of the oxygen consumption of the mitochondria for ATP production, the comparative group is less than the control group. This means that in the comparative group, the ATP production of the mitochondria under oxidative stress is decreased and the energy generated by the mitochondria is also decreased. In contrast, the experimental groups treated with the Lonicera Japonica extract of Examples 1 to 3 are higher than the comparative group and close to the control group. This means that the activity of the mitochondria in the experimental groups is enhanced by the Lonicera Japonica extract, and the ATP production of the mitochondria under oxidative stress in the experimental groups is also enhanced so that the mitochondria can generate enough energy for the cells so that can maintain the ATP production at the level close to the control group.

As shown in FIG. 6, in terms of the oxygen consumption of the mitochondria for the spare respiration, the comparative group is less than the control group. This means that in the comparative group, the spare respiration of the mitochondria under oxidative stress is decreased. In contrast, the experimental groups treated with the Lonicera Japonica extract of Examples 1 to 3 are higher than the comparative group and close to the control group. This means that the activity of the mitochondria in the experimental groups is enhanced by the Lonicera Japonica extract, and the spare respiration of the mitochondria under oxidative stress in the experimental groups is also enhanced. The enhancement of the spare respiration of the mitochondria means that the mitochondria have enhanced the ability of the mitochondria to cope with stress so that the mitochondria can maintain the spare respiration at the level close to the control group.

As shown in FIG. 7, in terms of the oxygen consumption of the mitochondria for the maximal respiration, the comparative group is less than the control group. This means that in the comparative group, the maximal respiration of the mitochondria under oxidative stress is decreased. In contrast, the experimental groups treated with the Lonicera Japonica extract of Examples 1 to 3 are higher than the comparative group and close to the control group. This means that the activity of the mitochondria in the experimental groups is enhanced by the Lonicera Japonica extract, and the maximal respiration of the mitochondria under oxidative stress in the experimental groups is also enhanced so that the mitochondria can maintain the maximal respiration at the level close to the control group.

As shown in FIG. 8, in terms of the ATP coupling efficiency, the comparative group is less than the control group, and the experimental groups treated with the Lonicera Japonica extract of Examples 1 to 3 are higher than the comparative group and close to the control group. This means that the activity of the mitochondria in the experimental groups is enhanced by the Lonicera Japonica extract, and the ATP coupling efficiency of the mitochondria under oxidative stress in the experimental groups is also enhanced so that the mitochondria can maintain the ATP coupling efficiency at the level close to those of the control group.

Bioenergetic health index (BHI) may be calculated by the oxygen consumption of the mitochondria according to the results analyzed by Seahorse XF analyzer. BHI is an index for evaluating the energy metabolism of the mitochondria calculated by the energy metabolism data of the mitochondria as parameters. BHI=log[(the oxygen consumption of mitochondria for ATP production)×(the oxygen consumption of the spare respiration)]/[(the oxygen consumption for overcoming proton leakage)×(the oxygen consumption of the non-mitochondrial respiration)]. The higher BHI of cells means the better activity of the mitochondria in the cells, as well as the better cellular resilience. Therefore, BHI may also be used as an indicator to evaluate the health of the mitochondria and the cells.

The BHI calculated by the above results of the oxygen consumption is shown in Table 3. As shown in Table 3, compared to the comparative group, the BHI of the mitochondria treated with the Lonicera Japonica extract of Examples 1 to 3 in the experimental groups is enhanced by the Lonicera Japonica extract. This means that the health of the mitochondria and the cells is also improved.

TABLE 3

| | Lonicera Japonica extract | |
|---|---|---|
| | (μg/mL) | BHI |
| Con. | — | 1.77 ± 0.03154 |
| Comp. | — | 0.91 ± 0.08980 |
| Ex. 1 | 200 | 1.68 ± 0.02247 |
| Ex. 2 | 250 | 1.69 ± 0.06771 |
| Ex. 3 | 500 | 1.72 ± 0.03790 |

According to the results described above, it can be seen that the Lonicera Japonica extract less than 500 μg/mL have no cytotoxicity. Also, the Lonicera Japonica extract can enhance the activity of the mitochondria. More specifically, the spare respiratory capacity, the maximal respiratory capacity and the ATP production of the mitochondria may be enhanced, the proton leakage of the mitochondria may be reduced, the ATP coupling efficiency of the mitochondria may be enhanced, and the BHI of the mitochondria may be enhanced.

In view of the above description, according to the present disclosure, referring to the use of extract in manufacturing a pharmaceutical or non-pharmaceutical composition for enhancing the activity of the mitochondria, wherein the extract is Lonicera Japonica extract, the Lonicera Japonica extract can enhance the spare respiratory capacity, the maximal respiratory capacity and the ATP production of the mitochondria, reduce the proton leakage of the mitochondria, enhance the ATP coupling efficiency of the mitochondria, and enhance the BHI of the mitochondria. The mitochondria whose activity is enhanced by the Lonicera Japonica extract can maintain their function and activity when being stressed to ensure normal work of cells without affecting by external or internal stress.

What is claimed is:

1. A use of an extract in manufacturing a pharmaceutical or non-pharmaceutical composition for enhancing the activity of mitochondria, wherein the extract is Lonicera Japonica extract.

2. The use of claim 1, wherein enhancing the activity of mitochondria comprises enhancing the spare respiratory capacity of the mitochondria.

3. The use of claim 1, wherein enhancing the activity of mitochondria comprises enhancing the maximal respiratory capacity of the mitochondria.

4. The use of claim 1, wherein enhancing the activity of mitochondria comprises enhancing the ATP production of the mitochondria.

5. The use of claim 1, wherein enhancing the activity of mitochondria comprises decreasing the proton leakage of the mitochondria.

6. The use of claim 1, wherein enhancing the activity of mitochondria comprises enhancing the ATP coupling efficiency of the mitochondria.

7. The use of claim 1, wherein enhancing the activity of mitochondria comprises enhancing the Bioenergetic Healthy Index (BHI) of the mitochondria.

8. The use of claim 1, wherein the Lonicera Japonica extract comprises chlorogenic acid.

9. The use of claim 8, wherein the concentration of the chlorogenic acid is 20 wt % to 30 wt % in the Lonicera Japonica extract.

10. The use of claim 1, wherein the Lonicera Japonica extract is obtained by extracting the petals of Lonicera Japonica with water and then drying, and the extraction volume ratio of the petals of Lonicera Japonica to water is 1:3.

11. The use of claim 1, wherein the concentration of the Lonicera Japonica extract is 200 μg/mL to 500 μg/mL in the composition.
